(19) United States
(12) Patent Application Publication
(10) Pub. No.: US 2019/0062391 A1
(43) Pub. Date: Feb. 28, 2019

(54) USE OF VEGF-B FOR TREATING DISEASES INDUCED BY OXIDATIVE INJURY

(71) Applicant: ZHONGSHAN OPHTHALMIC CENTER, SUN YAT-SEN UNIVERSITY, Guangzhou (CN)

(72) Inventors: Xuri Li, Guangzhou (CN); Zhongshu Tang, Guangzhou (CN)

(21) Appl. No.: 16/118,488

(22) Filed: Aug. 31, 2018

(30) Foreign Application Priority Data
Aug. 31, 2017 (CN) 201710776786.X

(51) Int. Cl. C07K 14/49; A61P 27/02

(52) U.S. Cl. CPC C07K 14/49; A61P 27/02

(57) ABSTRACT

The invention discloses a method for treating a disorder or a disease induced by an oxidative stress injury, including administering VEGF-B to a subject; and, a chemical containing VEGF-B protein, VEGF-B expressing plasmids, VEGF-B expressing viruses and/or VEGF-B expressing cells as active ingredients for treating a disorder or a disease induced by an oxidative stress injury. VEGF-B is a strong antioxidant having an intense anti-oxidative effect and capable of up-regulating antioxidant genes and down-regulating oxidant genes, and thus rescuing cellular and vascular degeneration under a pathologic condition. VEGF-B is the first member from the VEGF family to be recognized as a regulatory factor of anti-oxidation pathway, and therefore it can be used as a new medicament for anti-aging and anti-tumor purposes, and for treating varieties of degenerative and oxidative stress injury-related diseases.

### CROSS-REFERENCE TO RELATED APPLICATIONS

This present application claims the benefit of Chinese Patent Application No. 201710776786.X filed on Aug. 31, 2017, the contents of which are hereby incorporated by reference.

### FIELD OF THE INVENTION

The invention relates to the field of medical technology, particularly to a use of VEGF-B for treating a disorder or a disease induced by an oxidative stress injury.

### BACKGROUND OF THE INVENTION

Oxidative stress injury is a critical cause of aging, varieties of degenerative diseases and many other diseases. Due to that human body always contacts with external environment, free radicals are constantly generated inside the human body by factors including respiration (oxidative reaction), external pollutions and radiations. The free radicals or oxidants can break down cells and tissues, cause metabolic damage and thus cause varieties of health problems. Diseases closely related to oxidative stress injury include aging, degenerative diseases, inflammatory diseases, stroke, Alzheimer's disease, Parkinson's disease, Huntington's disease, cataracts, age-related macular degeneration (AMD), cancers, diabetes, diabetic retinal degeneration, arthritis, atherosclerosis, cardiovascular diseases, ischemia/reperfusion of myocardial or cerebral infarction, surgical hemostasis and the like.

Anti-oxidation can reduce and antagonize tissue damages caused by free radicals and oxidative stress. Studies indicate that anti-oxidation is a critical measure for preventing/slowing down aging. Meanwhile, the aforesaid diseases induced by free radicals and related to degeneration from aging can be prevented/slowed/treated by eliminating excessive oxidative free radicals. Therefore, as being able to effectively reduce the harm from oxidative stress injuries, antioxidant has a huge commercial potential, and thus becoming one of the main subjects for research and development in pharmaceutical, healthcare and cosmetic industries.

Primary pigmentary degeneration of retina, also called retinitis pigmentosa (RP), is a group of hereditary retinal degenerative diseases commonly represented by progressive loss of function of photosensory cells and pigmentary epithelium, and characterized by having major clinical symptoms including night blindness, progressive visual field damage, fundus pigmentation and abnormal or waveless electroretinogram. RP is also a common blinding eye disease worldwide, and millions of people suffer blindness resulting from retinal neurovascular degeneration.

The symptoms of RP commonly include a night blindness at early stage, peripheral visual field defect and damaged dark adaption afterwards, and finally a complete blindness. Photoreceptor cells of retina have vigorous metabolism and are thus extremely sensitive to oxidation. Degeneration of retinal arterioles can cause an increase in retinal apoptotic cells. Oxidative stress and vascular degeneration are considered the pathogenesis of RP and other neurodegenerative diseases, wherein 61 genes/loci are related to the occurrence of RP. Many mutations of genes related to the death of rod cells can result in peripheral retinal oxidative injuries, and thus it is challenging for scientists to correct the defective/mutated genes. As the neurodegenerative diseases are usually involved with complicated multi-pathogenic pathways, broad-spectrum treatments are much better than those specific for a single pathway in terms of rescuing the retinal photoreceptor cells and vasculature to restore eye functions.

It is known that there is currently no FDA-approved medicament for treating or preventing the progression of RP. For RP patients, despite that the progression of RP can be slowed by administrating nutritional supplements comprising antioxidants, such exogenous treatment cannot achieve satisfying and lasting therapeutic effect. Moreover, no endogenous antioxidant defense agent to prevent or delay degeneration is currently in active development.

VEGF-B is a member of the VEGF (vascular endothelial growth vector) family and expressed in most tissues and organs. Unlike other members of the VEGF family, VEGF-B does not affect neoangiogenesis and vascular permeability. Both isoforms—VEGF-B₁₆₇, a strong heparan sulfate binder and VEGF-B₁₈₆, a soluble isoform, bind to neuropilin-1 and VEGF-receptor 1, which are expressed mainly in vascular endothelial cells. VEGF-B₁₆₇ is also a strong protective/survival factor of various neurons, which promotes neural/vascular survival by regulating the expression of many vascular survival-promoting genes via NP-1 and VEGFR-1. However, the molecular mechanism and activity of VEGF-B₁₈₆ remains unclear.

### SUMMARY OF THE INVENTION

During the study of the molecular mechanism and function of VEGF-B, the inventors of the present invention surprisingly found new uses of VEGF-B.

As a first aspect, the invention provides a method of treating a disorder or a disease induced by an oxidative stress injury in a subject, comprising administering VEGF-B to the subject. It is to be noted that the disorder or the disease includes sports fatigue, heel pain, lower extremity weakness, mental fatigue and the like.

Preferably, the VEGF-B is administered in the form of VEGF-B protein, VEGF-B expressing plasmids, VEGF-B expressing viruses and/or VEGF-B expressing cells.

Preferably, the VEGF-B is VEGF-B₁₆₇ and/or VEGF-B₁₈₆.

Preferably, the VEGF-B is a modified VEGF-B, the modified VEGF-B is a cyclized, phosphorylated and/or methylated VEGF-B; or the VEGF-B is a recombinant protein or polypeptide having 1-5 more or less amino acids than the VEGF-B.

Preferably, the disorder or the disease induced by the oxidative stress is selected from a cancer, a degenerative disease, inflammation, stroke, Alzheimer's disease, Parkinson's disease, Huntington's disease, cataract, age-related macular degeneration (AMD), diabetes, diabetic retinal degeneration, arthritis, atherosclerosis and a cardiovascular disease.

Preferably, the disorder or the disease induced by the oxidative stress is a degenerative disease.

Preferably, the degenerative disease is retinitis pigmentosa; more preferably, the VEGF-B treats the retinitis pigmentosa by inhibiting retinal tissue degeneration and retinal vascular degeneration.

Preferably, the VEGF-B inhibits the retinal tissue degeneration and the retinal vascular degeneration by up-regulating the expression of antioxidant genes and survival-related genes, and/or by down-regulating the expression of oxidative stress genes and apoptosis-related genes.

Preferably, the antioxidant genes comprise Gpx1, Sod1, Prdx5, Prdx6-rs1, Txnrd3, Sod2, Gpx5, Zmynd17, Gpx2, Txnrd1, Prdx1, Gpx6 and Gsr; the survival-related genes comprise SGK1, GDNF, CNTG, MDM4, SKP2, UNC5C, BDNF, Ubqln1 and CDNF, the oxidative stress genes comprise Ptgs1, Nox4, Ncf2, Tpo and Ppp1r15b; and the apoptosis-related genes comprise Bid, Bax, Bik, Bad and Pmaip.

As a second aspect, the invention further provides a chemical comprising VEGF-β protein, VEGF-B expressing plasmids, VEGF-B expressing viruses and/or VEGF-B expressing cells as active ingredients to treat a disorder or a disease induced by an oxidative stress injury.

Preferably, the chemical is a pharmaceutical composition, an antioxidant, a healthcare product or an anti-aging product;

Preferably, the disorder or the disease induced by the oxidative stress is selected from a cancer, a degenerative disease, inflammation, stroke, Alzheimer's disease, Parkinson's disease, Huntington's disease, cataract, age-related macular degeneration (AMD), diabetes, diabetic retinal degeneration, arthritis, atherosclerosis and a cardiovascular disease.

In summary, the advantages of the invention are as follows:

VEGF-B is a strong antioxidant having an intense anti-oxidative effect which can up-regulate antioxidant genes and down-regulate oxidant genes, and thus rescue the photoreceptor cells and blood vessels from degeneration under a pathologic condition. VEGF-B is the first member from the VEGF family to be recognized as a regulatory factor of anti-oxidation pathway, and therefore it can be used as a new medicament for anti-aging and anti-tumor purposes, and for treating varieties of degenerative diseases.

### BRIEF DESCRIPTION OF THE DRAWINGS

FIGS. 1A-1C show the results of retinal degeneration caused by VEGF-B deficiency in Experiment 3 of the present invention; wherein:

FIG. 1A shows the result of HE staining of retinae from wild type mice (left, WT) and VEGF-B₁₆₇ deficient mice (right, VEGF-B −/−);the retinal layers marked include RGC (retinal ganglion cells), IPL (inner plexiform layer), INL (inner nuclear layer), ONL (outer nuclear layer), IS/OS (inner/outer photoreceptor segment) and Ch (chorioid);

FIG. 1B shows the total retinal thickness in FIG. 1A;

FIG. 1C shows the thickness of each retinal layer in FIG. 1A; The retinal layers marked include IPL (inner plexiform layer), INL (inner nuclear layer), OPL (outer plexiform layer), ONL (outer nuclear layer) and IS/OS (inner/outer photoreceptor segment);

FIGS. 2A-2E show the results of retinal atrophy caused by VEGF-B deficiency in Experiment 3 of the present invention; wherein:

FIG. 2A shows the result of HE staining of retinae from wild type mice (left, WT) and VEGF-B₁₆₇ deficient mice (right, VEGF-B −/−); the retinal layers marked include RGC (retinal ganglion cells), IPL (inner plexiform layer), INL (inner nuclear layer), ONL (outer nuclear layer), IS/OS (inner/outer photoreceptor segment) and Ch (chorioid);

FIG. 2B shows the total retinal thickness in FIG. 2A;

FIG. 2C shows the thickness of each retinal layer in FIG. 2A; the retinal layers marked include RGC (retinal ganglion cells), IPL (inner plexiform layer), INL (inner nuclear layer), OPL (outer plexiform layer), ONL (outer nuclear layer), IS/OS (inner/outer photoreceptor segment) and Ch (chorioid);

FIG. 2D shows the result of TUNEL staining of retinae from the wild type mice injected with IgG (left) and the wild type mice injected with VEGF-B₁₆₇ neutralizing antibody (right, VEGF-B nab);

FIG. 2E shows the rate of TUNEL-positive field in FIG. 2D.

FIGS. 3A-3G show the results of Experiment 4 of the present invention; wherein:

FIG. 3A shows the result of HE staining of retinae from rd1 mice injected with BSA (left) or VEGFB₁₆₇ (right, VEGF-B); the retinal layers marked include RGC (retinal ganglion cells), IPL (inner plexiform layer), INL (inner nuclear layer) and ONL (outer nuclear layer);

FIG. 3B shows the total retinal thickness in FIG. 3A;

FIG. 3C shows the thickness of each retinal layer in FIG. 3A; the retinal layers marked include RGC (retinal ganglion cells), OPL (outer plexiform layer), ONL (outer nuclear layer), IPL (inner plexiform layer) and INL (inner nuclear layer);

FIG. 3D shows the result of TUNEL staining of retinae from the rd1 mice injected with BSA (left) or VEGF-B₁₆₇ (right, VEGF-B), wherein the arrows indicate TUNEL-positive field;

FIG. 3E shows the rate of TUNEL-positive field in FIG. 3D;

FIG. 3F shows the result of Real-time qPCR detecting the expression level of apoptosis-related genes;

FIG. 3G shows the result of Real-time qPCR detecting the expression level of survival-related genes.

FIGS. 4A-4G show the results of Experiment 5 of the present invention; wherein:

FIG. 4A shows the result of IB4 staining of retinae from rd1 mice injected with BSA (left) or adeno-associated viruses carrying VEGF-B₁₆₇ (right, VEGF-B); the retinal layers marked include RGC (retinal ganglion cells), IPL (inner plexiform layer), INL (inner nuclear layer) and ONL (outer nuclear layer);

FIG. 4B shows the density of retinal blood vessels of rd1 mice injected with BSA or adeno-associated viruses carrying VEGF-B₁₆₇;

FIG. 4C shows the result of immunofluorescence staining of rhodopsin of retinae from the rd1 mice injected with BSA (left) or adeno-associated viruses carrying VEGF-B₁₆₇ (right, VEGF-B), wherein the arrows indicate rhodopsin-positive fields; wherein the

FIG. 4D shows the rate of rhodopsin-positive field in FIG. 4C;

FIG. 4E shows the expression of rhodopsin in the retinae in FIG. 4C;

FIG. 4F shows the result of immunofluorescence staining of PNA (peanut agglutinin) of retinae from the rd1 mice injected with BSA (left) or adeno-associated viruses carrying VEGF-B₁₆₇ (right, VEGF-B), wherein the arrows indicate PNA-positive fields;

FIG. 4G shows the rate of PNA-positive field in FIG. 4F.

FIGS. 5A-5D show the results of Experiment 6 of the present invention; wherein:

FIG. 5A shows the expression of various genes in the retinae from rd1 mice injected with BSA, VEGF-B₁₆₇, GPX-1 shRNA or control shRNA;

FIG. 5B shows the result of western blot detecting the protein expression of GPX-1 and SOD-1 in retinae of the rd1 mice injected with VEGF-B₁₆₇ or GPX-1 shRNA;

FIG. 5C shows the cell survival rate of 1% H₂O₂-treated 661W cells rescued by IgG or VEGF-B₁₆₇;

FIG. 5D shows the cell survival rate of 0.5% H₂O₂-treated RPE cells rescued by IgG or VEGF-B₁₆₇.

FIGS. 6A-6E show the results of Experiment 7 of the present invention; wherein:

FIG. 6A shows the result of western blot detecting the protein expression level of VEGF-B in eyes of rd1 mice injected with GFP-AAV (control vector) or VEGF-B₁₆₇-AAV;

FIG. 6B shows the result of HE staining of retinae from the rd1 mice injected with GFP-AAV (left, GFP) or VEGF-B₁₆₇-AAV (right, AAV2+VEGF-B₁₆₇); the retinal layers marked include RFL (retinal fiber layer), GCL (ganglion cells layer), IPL (inner plexiform layer), INL (inner nuclear layer), ONL (outer nuclear layer) and RPE/Ch (retinal pigment epithelium/chorioid);

FIG. 6C shows the total retinal thickness, and thickness of each retinal layer in FIG. 6B; the retinal layers marked include GCL (ganglion cells layer), IPL (inner plexiform layer), INL (inner nuclear layer), OPL (outer plexiform layer), ONL (outer nuclear layer) and RPE/Ch (retinal pigment epithelium/chorioid);

FIG. 6D shows the result of immunofluorescence staining of rhodopsin of retinae from the rd1 mice injected with GFP-AAV (left) or VEGF-B₁₆₇-AAV (right); the retinal layer marked is ONL (outer nuclear layer);

FIG. 6E shows rhodopsin positive rate in each retinal regions in FIG. 6D; the regions marked include superior regions 1-3 (S1, S2, S3) and inferior regions 3-1 (I3, I2, I1).

FIGS. 7A-7H show the results of Experiment 8 of the present invention; wherein:

FIG. 7A shows the result of HE staining of retinae from rd1 mice (left, rd1) and VEGF-B₁₈₆ transgenic mice (right, VEGF-B₁₈₆-Rho-Tg);

FIG. 7B shows the total retinal thickness and thickness of each retinal layer in FIG. 7A;

FIG. 7C shows the result of western blot detecting the protein expression level of VEGF-B in the rd1 mice and VEGF-B₁₈₆ mice (VEGF-B₁₈₆-Rho-Tg);

FIG. 7D shows the result of Real-time qPCR detecting the expression level of rhodopsin;

FIG. 7E shows the result of western blot detecting the protein expression level of rhodopsin;

FIG. 7F shows the result of TUNEL staining of retinae from the rd1 mice (left, rd1) and VEGF-B₁₈₆ transgenic mice (right, VEGF-B₁₈₆-Rho-Tg);

FIG. 7G shows the vascular density in FIG. 7F;

FIG. 7H shows the rate of TUNEL-positive regions; the regions marked include superior regions 1-3 (S1, S2, S3) and inferior regions 3-1 (I3, I2, I1).

### DETAILED DESCRIPTION OF ILLUSTRATED EMBODIMENTS

In order to better illustrate the purpose, technical solutions and advantages of the invention, the invention will be further illustrated in conjunction with the drawings and experiments.

### Experiment 1: VEGF-B₁₆₇ Up-Regulates the Expression of Antioxidant Genes

The retinal neuro/blood vessel degenerative rd1 (FVB/NJ) mice commonly used as a model for retinitis pigmentosa were obtained from the Jackson Laboratory (Bar Harbor, Me., USA). The characteristics of rd1 mice is single row of thin PR nuclei remains, which are surviving cones not the rods at P21, leading to a complete loss of night vision. Total RNA was extracted from retinae of rd1 mice treated with VEGF-B₁₆₇ or BSA (as control). 1 μg of the total RNA was reversely transcribed to cDNA, and a high throughput RT²PCR was performed to detect the expression of varieties of antioxidant genes. The expression of antioxidant genes are listed in Table 1. A microarray assay was further performed on HUSMC (human uterine smooth muscle cells) treated with VEGF-B₁₆₇ or BSA (as control).

TABLE 1
Expression of antioxidant genes

| | | Fold of up-regulation | |
|---|---|---|---|
| | | PCR array | Microarray |
| Gene ID | Gene name | (retina of rd1 mice) | (HUSMC) |
| NM_008160 | Gpx1 | 8.6 | 3.9 |
| NM_011434 | Sod1 | 8.3 | 2.0 |
| NM_012021 | Prdx5 | 5.6 | 2.8 |
| NM_177256 | Prdx6-rs1 | 5.4 | 1.6 |
| NM_153162 | Txnrd3 | 4.5 | 1.8 |
| NM_013671 | Sod2 | 4.4 | 2.0 |
| NM_010343 | Gpx5 | 4.2 | 1.9 |
| NM_029104 | Zmynd17 | 4.0 | 2.0 |
| NM_030677 | Gpx2 | 2.9 | 1.6 |
| NM_015762 | Txnrd1 | 2.8 | 3.7 |
| NM_011034 | Prdx1 | 2.8 | 2.0 |
| NM_145451 | Gpx6 | 2.3 | NC |
| NM_010344 | Gsr | 2.0 | 2.3 |

Table. 1 shows that Gpx1 and other antioxidant genes were up-regulated by 2.0-8.6 folds after the VEGF-B ₁₆₇ treatment.

The result of the microarray assay was consistent with that from the rd1 mice.

### Experiment 2: VEGF-B₁₆₇ Inhibits the Expression of Oxidative Stress Genes

Total RNA was extracted from retinae of rd1 mice treated with VEGF-B₁₆₇ or BSA (as control). 1 μg of the total RNA was reversely transcribed to cDNA, and a high throughput RT²PCR was performed to detect the expression of varieties of oxidative stress genes. The expression of oxidative stress genes are listed in Table 2. A microarray assay was further performed on HUSMC (human uterine smooth muscle cells) treated with VEGF-B₁₆₇ or BSA (as control).

TABLE 2
Expression of oxidative stress genes

| | | Fold of down-regulation | |
|---|---|---|---|
| | | PCR array | Microarray |
| Gene ID | Gene name | (retina of rd1 mice) | (HUSMC) |
| NM_008969 | Ptgs1 | 2.3 | NC |
| NM_015760 | Nox4 | 2.2 | 2.3 |
| NM_010877 | Ncf2 | 2.1 | 2.1 |
| NM_009417 | Tpo | 2.1 | 1.7 |
| NM_133819 | Ppp1r15b | 2.0 | 2.0 |

Table. 2 shows that Ptgs1 and other oxidative stress genes were down-regulated by 2.0-2.3 folds after the VEGF-B₁₆₇ treatment.

The result of the microarray assay was consistent with that from the rd1 mice.

### Experiment 3: VEGF-B₁₆₇ Deficiency Causes Retinal Degeneration and Atrophy

Eyes were separated from VEGF-B₁₆₇ deficient mice and littermate wild type mice. The eyes were embedded in OCT, sectioned and fixed, and were stained by HE for measuring retinal thickness. The wild type mice were intravitreally injected with VEGF-B₁₆₇ neutralizing antibody or IgG (2 μ/eye). Eyes were separated and sectioned one week later, apoptotic cells were detected using a TUNEL kit.

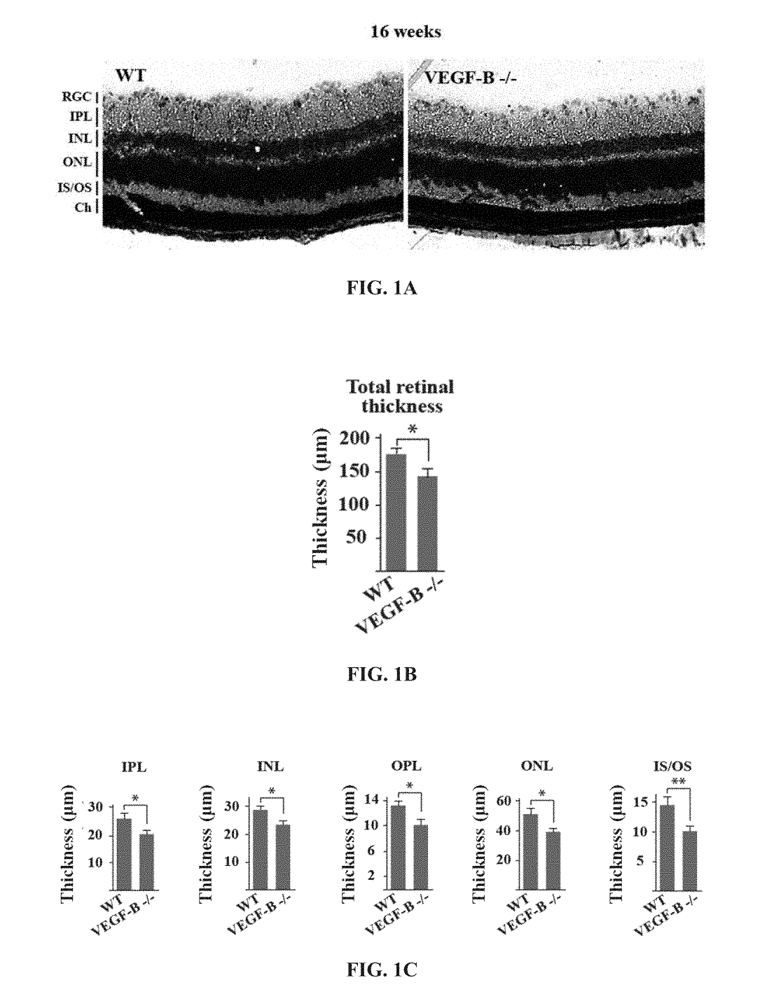

FIG. 1A shows and points out different retinal layers after the HE staining, including RGC (retinal ganglion cells), IPL (inner plexiform layer), INL (inner nuclear layer), ONL (outer nuclear layer), IS/OS (inner/outer photoreceptor segment) and Ch (chorioid). As illustrated in FIGS. 1A-1C, compared with those of the wild type mice, the total retinal thickness (FIG. 1B) as well as the thickness of each retinal layer (i.e., IPL, INL, OPL, ONL, IS/OS) (FIG. 1C) significantly attenuated in the VEGF-B₁₆₇ deficient mice. This indicates that VEGF-B₁₆₇ deficiency causes retinal degeneration.

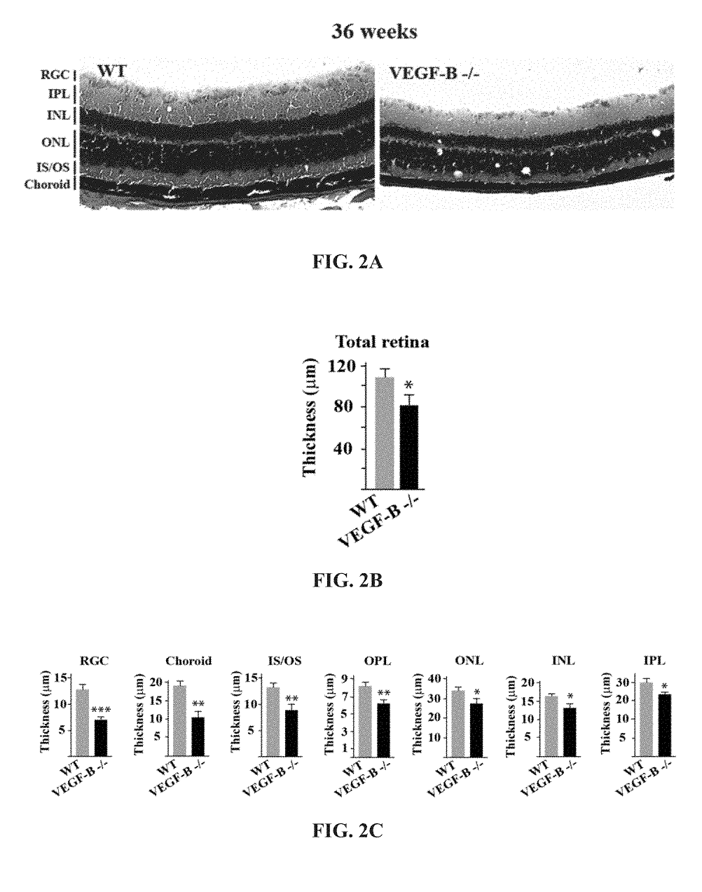

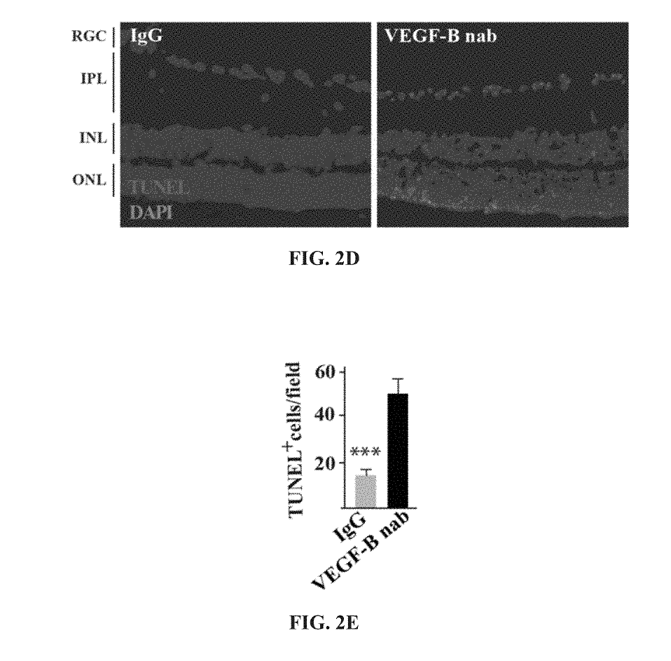

FIG. 2A shows and points out different retinal layers after the HE staining. As illustrated in FIGS. 2A-2E, compared with those of the wild type mice, the total retinal thickness (FIG. 2B) as well as the thickness of each retinal layer (i.e., RGC, Ch, IS/OS, OPL, ONL, INL, IPL) (FIG. 2C) significantly attenuated in the VEGF-B₁₆₇ deficient mice. This indicates that VEGF-B₁₆₇ deficiency causes retinal atrophy.

FIG. 2D shows and points out different retinal layers after the TUNEL staining. The results show that the inhibition on VEGF-B₁₆₇ from the neutralizing antibody caused retinal cell apoptosis (FIG. 2E) in wild type mice.

### Experiment 4: VEGF-B₁₆₇ Treatment Repairs Degenerated Retinae of rd1 Mice

The retinal neural/blood vessel degenerative rd1 (FVB/NJ) mice commonly used as a model for retinitis pigmentosa were obtained from the Jackson Laboratory (Bar Harbor, Me, USA). The characteristics of rd1 mice is single row of thin PR nuclei remains, which are surviving cones not the rods at P21, leading to a complete loss of night vision. The 11-day-old rd1 mice were intravitreally injected with VEGF-B₁₆₇ or equivalent BSA (500 ng/eye), the injection was repeated every 5 days for twice later. The mice were euthanized 26 days after birth, the vitreous bodies thereof were separated, embedded and sectioned, and were stained by HE for measuring retinal thickness. Apoptotic cells were detected using a TUNEL kit. A real-time PCR was performed to detect gene expression.

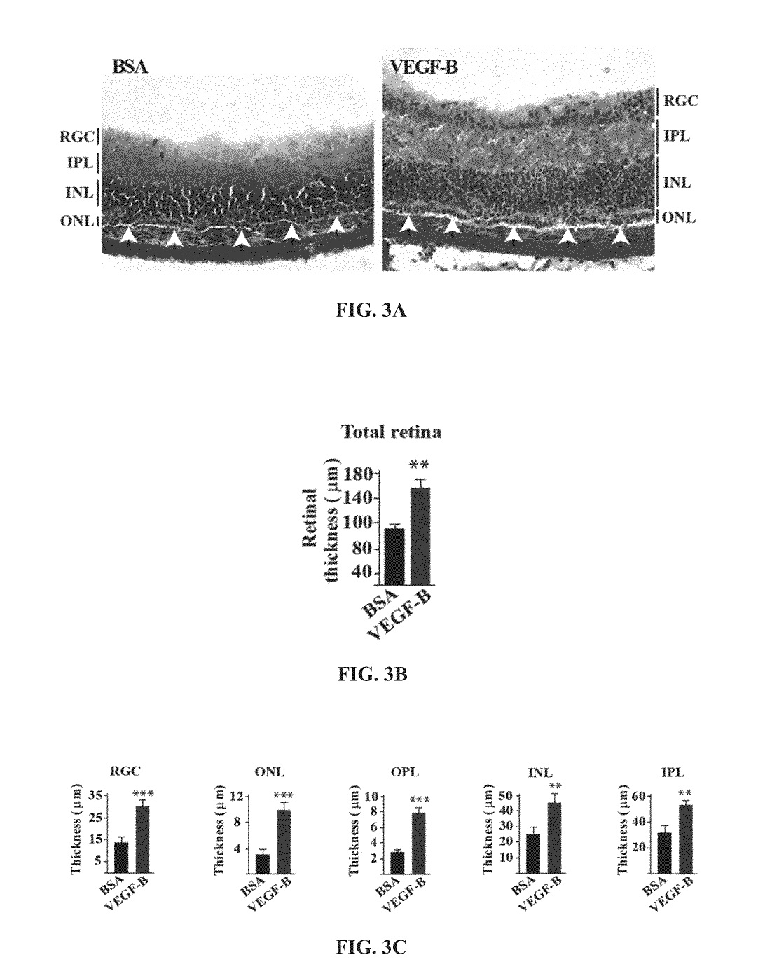

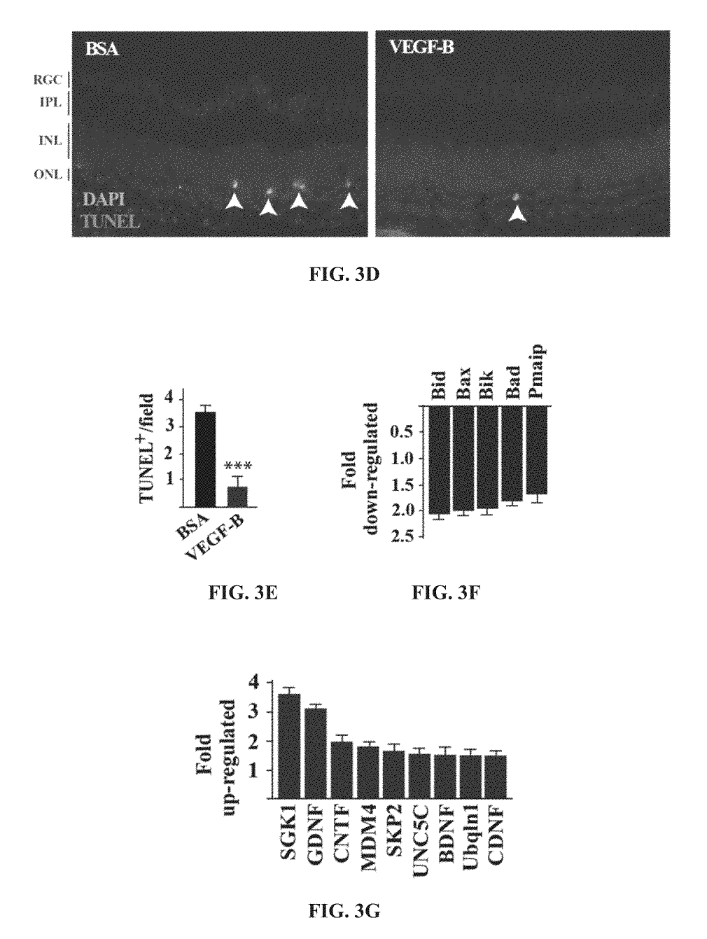

FIG. 3A shows and points out different retinal layers after the HE staining. The VEGF-B₁₆₇ treatment alleviated the retinal degeneration of the rd1 mice (FIGS. 3A, 3B), while each retinal layer was also significantly thickened (FIG. 3C).

FIG. 3D shows and points out different retinal layers after the TUNEL staining. TUNEL staining shows that compared with the control, the rd1 mice treated with VEGF-B₁₆₇ had significantly decreased apoptotic cells (FIG. 3E).

Result of Real-time PCR shows that the expression of apoptosis-related genes were down-regulated (FIG. 3F) and survival-related genes were up-regulated (FIG. 3G) in the mice after the VEGF-B₁₆₇ treatment.

### Experiment 5: VEGF-B₁₆₇ Increases the Survival Rate of Rod Cells and Cone Cells of rd1 Mice

The rd1 mice were intravitreally injected with adeno-associated viruses (AAV) carrying VEGF-B₁₆₇ (GFP was used as control), as to overexpress VEGF-B₁₆₇ in eyes. The eyeballs were frozenly sectioned and stained by IB4. An immunofluorescence staining of rhodopsin was performed. A real-time PCR was performed to detect the expression of rhodopsin. An immunofluorescence staining of peanut agglutinin (PNA) was performed.

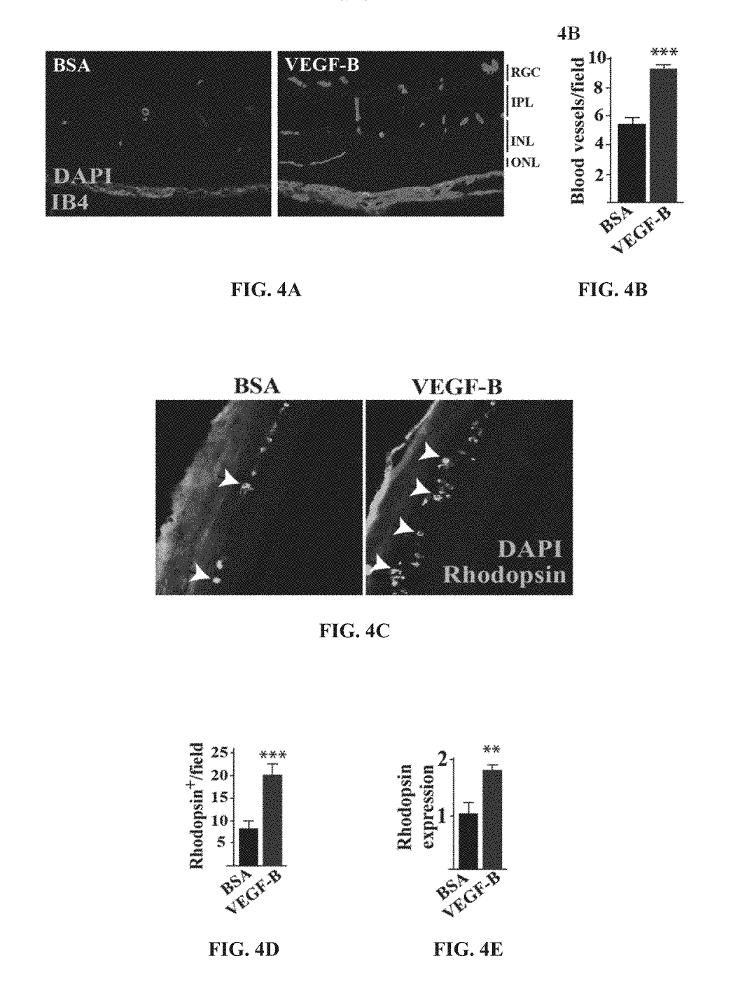

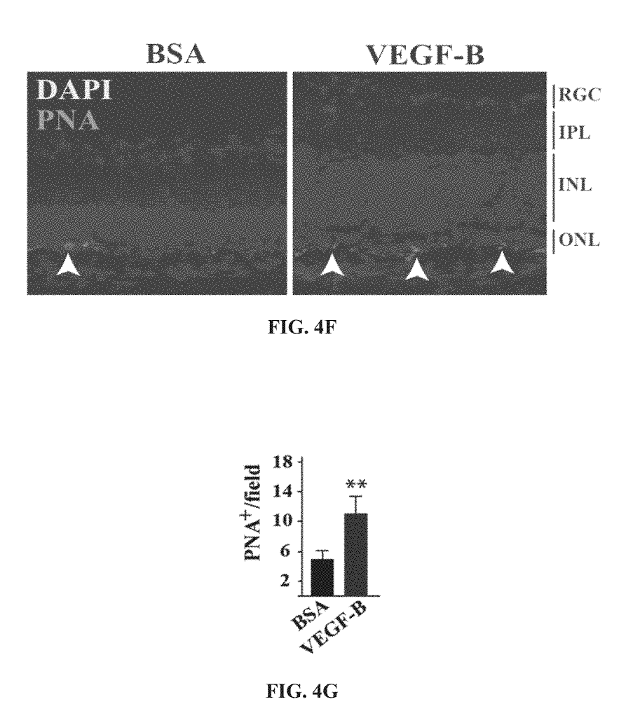

FIG. 4A shows and points out different retinal layers after the IB4 staining. Result shows that the VEGF-B₁₆₇-overexpressing mice had increases in retinal thickness (FIG. 4A) and vascular area (FIG. 4B).

FIG. 4C shows and points out rhodopsin positive sites in retina after the immunofluorescence staining of rhodopsin. The result shows that compared with the control, the VEGF-B₁₆₇-overexpressing mice had a significant increase in rhodopsin-positive rod cells (FIG. 4D).

Result of Real-time PCR confirms that the VEGF-B₁₆₇-overexpressing mice had an increase in rhodopsin expression in the mRNA level (FIG. 4E).

Immunofluorescence staining of PNA shows that the VEGF-B₁₆₇-overexpressing mice had an increase in PNA-positive cone cells, indicating that VEGF-B₁₆₇ has protective effect on both photoreceptor cone cells and rod cells (FIGS. 4F, 4G).

### Experiment 6: Gpx-1 shRNA Eliminates the Effect of VEGF-B₁₆₇

The rd1 mice were intravitreally injected with VEGF-B₁₆₇expressing AAV, and were simultaneously injected with Gpx-1 shRNA or control shRNA. Retinae were extracted and processed. A Quantitative PCR was performed on the retina samples to detect the expression of Sod-1/Zmynd17/Prdx-1/Prdx -5/Gpx-2/TPO gene. A western blot was performed on such retina samples to detect the protein expression level of GPX1 and SOD1. 661W photoreceptor cells were cultured in DMEM medium added with 10% FBS and antibiotics. The cells were treated with 1% H₂O₂, and then added with VEGF-B₁₆₇. RPE cells were cultured in F12K medium added with 10% FBS and antibiotics. The cells were treated with 0.5% H₂O₂, and then added with VEGF-B₁₆₇.

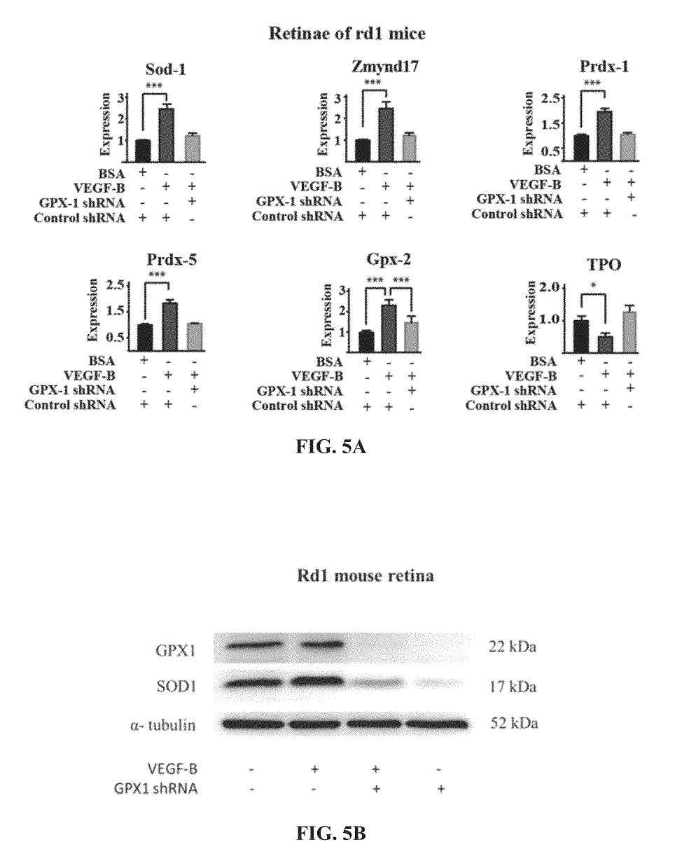

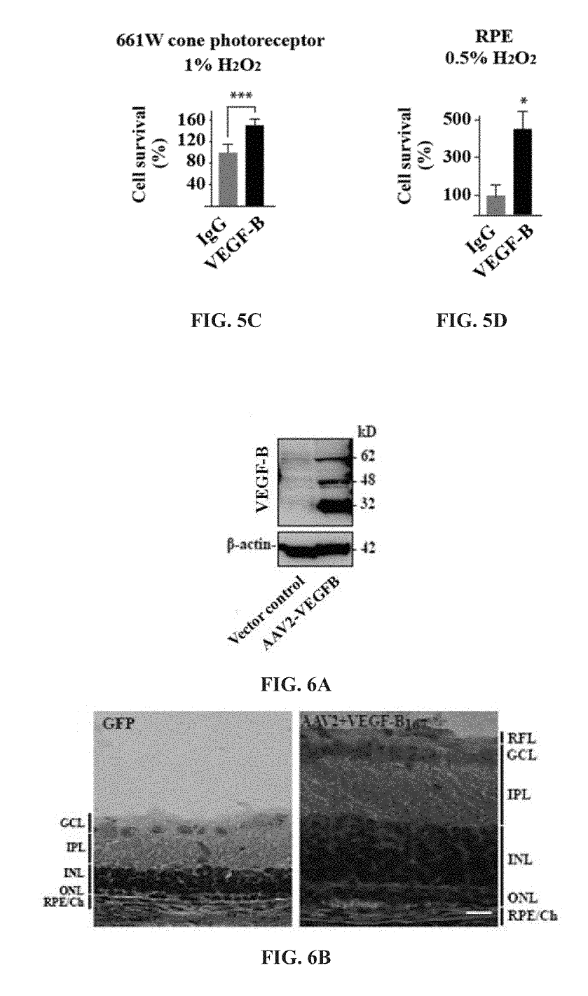

As illustrated in FIGS. 5A-5D, result shows that Gpx-1 shRNA eliminated the up-regulation of Sod-1/Zmynd17/Prdx-1/Prdx-5/Gpx-2 gene and the down-regulation of oxidative stress gene Tpo from VEGF-B₁₆₇ (FIG. 5A). Result of western blot confirmed such result from the protein level (FIG. 5B).

The result also shows that VEGF-B₁₆₇ significantly promoted cell survival for 661W cells (FIG. 5C) and RPE cells (FIG. 5D) which were treated with H₂O₂.

### Experiment 7: Protective Effect of AAV (Adeno-Associated Viruses) -Expressed VEGF-B₁₆₇ on Retina

Eyes of rd1 mice were injected with VEGF-B₁₆₇ expressing AAV or GFP-AAV (as control). The eyes were fixed, embedded and sectioned, and were stained by HE for measuring retinal thickness. An immunofluorescence staining of rhodopsin was performed. A western blot was performed on such eye samples to detect the protein expression level of VEGF-B.

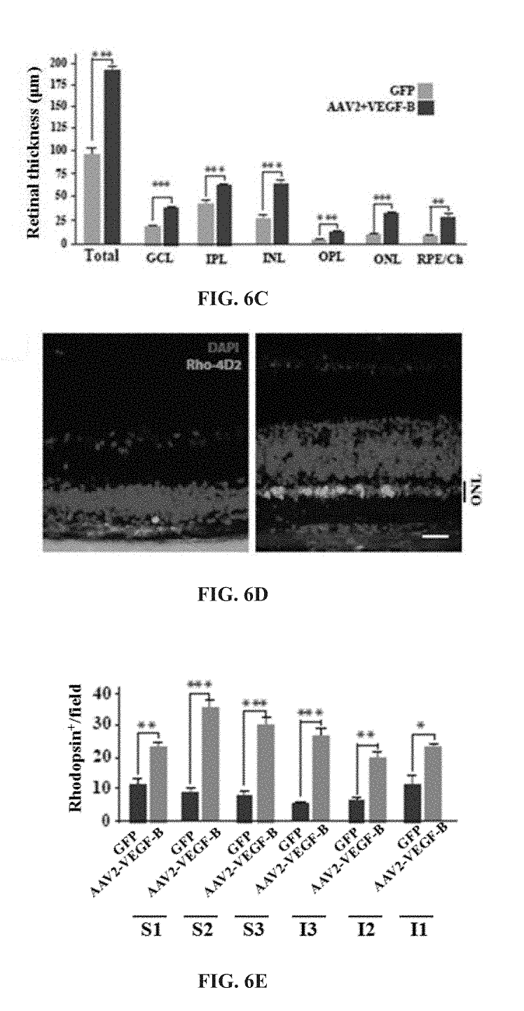

As illustrated in FIGS. 6A-6E, result of Western blot shows the expression of VEGF-B in the eyes of rd1 mice injected with VEGF-B₁₆₇-AAV (FIG. 6A). Compared with GFP-AAV control, the rd1 mice injected with VEGF -B₁₆₇-AAV had increases in both total retinal thickness (FIG. 6B) and thickness of each retinal layer (FIG. 6C), and in the amount of rhodopsin-positive cells (FIGS. 6D, 6E).

### Experiment 8: VEGF-B₁₈₆-Transgenic Mice have Therapeutic Effect on Retinal and Vascular Degeneration

In order to investigate the effect of VEGF-B₁₈₆ isoform, a rod cells -specific VEGF-B₁₈₆ transgenesis was performed on rd1 mice for an overexpression of VEGF-B₁₈₆ in rod cells, while rd1 mice were used as control. Retinae of the transgenic mice and the rd1 mice were separated, fixed, embedded and sectioned, and were stained by HE for measuring retinal thickness. A western blot and quantitative PCR were performed on such retina samples from the transgenic mice and the rd1 mice to detect the expression level of VEGF-B and rhodopsin. An immunofluorescence staining of rhodopsin was performed.

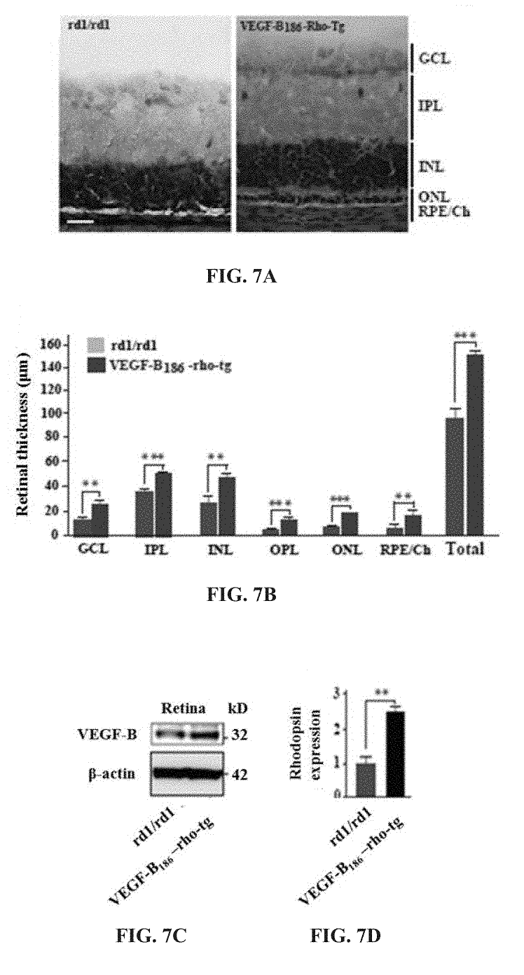

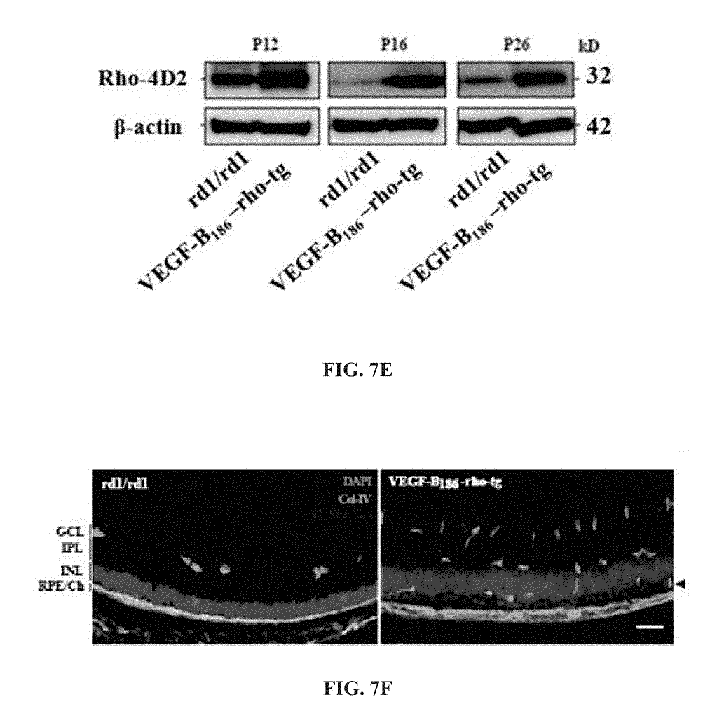

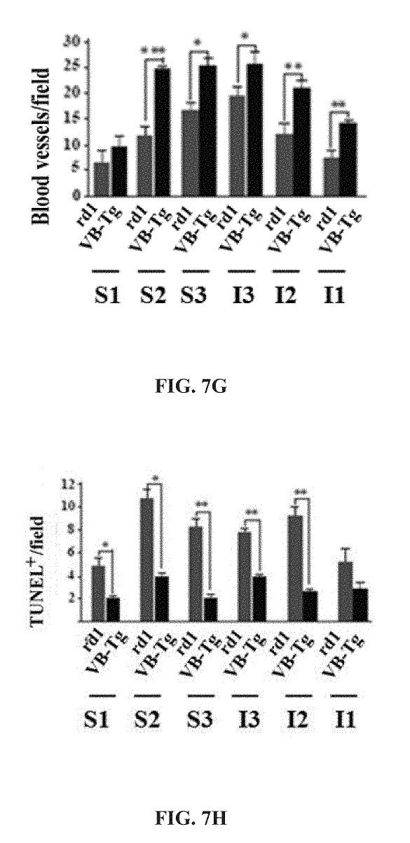

As illustrated in FIGS. 7A-7H, result shows that compared with control, the VEGF-B₁₈₆-overexpressing mice had significant increases in both total retinal thickness (FIG. 7A) and thickness of each retinal layer (FIG. 7B). Result of Western blot shows an increase in retinal VEGF-B₁₈₆ expression (FIG. 7C). Results of quantitative PCR (FIG. 7D) and Western blot (FIG. 7E) confirm an increase in retinal rhodopsin expression. The result also shows a decrease in TUNEL-positive (apoptotic) cells (FIGS. 7F-7H) and an increase in vascular density (FIG. 7G). In summary, all of the aforesaid results confirm that VEGF -B₁₈₆ has therapeutic effect on retinal and vascular degeneration.

It is to be noted that, the embodiments disclosed above are only used to illustrate the technical scheme of the invention, not to limit the scope of the invention. Despite that the illustration is made in reference to the preferred embodiments, those skilled in the art should understand that many improvements and alternatives can be made without departing from the principle of the invention, these improvements and alternatives should also be included in the scope of the invention.

1. A method of treating a disorder or a disease induced by an oxidative stress injury in a subject, comprising:
administering VEGF-B to the subject.

2. The method according to claim 1, wherein the disorder or the disease induced by the oxidative stress injury is selected from a cancer, a degenerative disease, inflammation, stroke, Alzheimer's disease, Parkinson's disease, Huntington's disease, cataract, age-related macular degeneration, diabetes, diabetic retinal degeneration, arthritis, atherosclerosis and a cardiovascular disease.

3. The method according to claim 1, wherein the disorder or the disease induced by the oxidative stress is a degenerative disease.

4. The method according to claim 3, wherein the degenerative disease is retinitis pigmentosa.

5. The method according to claim 4, wherein the VEGF-B treats the retinitis pigmentosa by inhibiting retinal tissue degeneration and retinal vascular degeneration.

6. The method according to claim 5, wherein the VEGF-B inhibits the retinal tissue degeneration and the retinal vascular degeneration by up -regulating the expression of antioxidant genes and survival-related genes, and/or by down-regulating the expression of oxidative stress genes and apoptosis -related genes.

7. The method according to claim 6, wherein the antioxidant genes comprise Gpx1, Sod1, Prdx5, Prdx6-rs1, Txnrd13, Sod2, Gpx5, Zmynd17, Gpx2, Txnrd1, Prdx1, Gpx6 and Gsr; the survival-related genes comprise SGK1, GDNF, CNTG, MDM4, SKP2, UNC5C, BDNF, Ubqln1 and CDNF; the oxidative stress genes comprise Ptgs1, Nox4, Ncf2, Tpo and Ppp1r15b; and the apoptosis-related genes comprise Bid, Bax, Bik, Bad and Pmaip.

8. The method according to claim 1, wherein the VEGF-B is administered in the form of VEGF-B protein, VEGF-B expressing plasmids, VEGF-B expressing viruses and/or VEGF-B expressing cells.

9. The method according to claim 1, wherein the VEGF-B is VEGF-B₁₆₇ and/or VEGF-B₁₈₆.

10. The method according to claim 1, wherein the VEGF-B is a modified VEGF-B, the modified VEGF-B is a cyclized, phosphorylated and/or methylated VEGF-B; or the VEGF-B is a recombinant protein or polypeptide having 1-5 more or less amino acids than the VEGF-B.

11. A chemical comprising VEGF-B protein, VEGF-B expressing plasmids, VEGF-B expressing viruses and/or VEGF-B expressing cells as active ingredients to treat a disorder or a disease induced by an oxidative stress injury.

12. The chemical according to claim 11, wherein the chemical is a pharmaceutical composition, an antioxidant, a healthcare product or an anti -aging product.
